(19) United States
(12) Patent Application Publication
(10) Pub. No.: US 2019/0076510 A1
(43) Pub. Date: Mar. 14, 2019

(54) PROTEIN KINASE C INHIBITION TO EXTEND TISSUE PLASMINOGEN ACTIVATOR TREATMENT FOR ISCHEMIC DISEASE

(71) Applicant: THE REGENTS OF THE UNIVERSITY OF MICHIGAN, Ann Arbor, MI (US)

(72) Inventors: Daniel A. Lawrence, Ann Arbor, MI (US); David A. Antonetti, Ann Arbor, MI (US)

(21) Appl. No.: 16/129,870

(22) Filed: Sep. 13, 2018

Related U.S. Application Data
(60) Provisional application No. 62/558,146, filed on Sep. 13, 2017.

(51) Int. Cl. A61K 38/49; A61K 31/407; A61P 9/10

(52) U.S. Cl. CPC A61K 38/49; A61P 9/10; A61K 31/407; C12Y 304/21068

(57) ABSTRACT

The disclosure provides methods for extending the time post-ischemia during which a tissue plasminogen activator compound can be efficaciously and safely administered to a subject with an ischemic disease such as stroke.

### CROSS-REFERENCE TO RELATED APPLICATIONS

This application claims priority to U.S. Provisional Application No. 62/558,146, filed Sep. 13, 2017, which is hereby incorporated by reference in its entirety.

### STATEMENT OF GOVERNMENT INTEREST

This invention was made with government support under EY012021, EY023725, HL055374, and NS079639 awarded by the National Institutes of Health. The government has certain rights in the invention.

### FIELD

The disclosed subject matter generally relates to the field of the medical treatment of disease and, more specifically, to the treatment of ischemic disease.

### BACKGROUND

Protein kinase c inhibitors have been developed by pharmaceutical companies to control vascular permeability in blinding eye diseases such as diabetic retinopathy. These compounds include Ruboxistaurin®, developed by Eli Lilly. These compounds were shown to reduce vascular permeability and edema in the eye.

Stroke remains a leading cause of morbidity and mortality with limited therapeutic options. The current standard of care for patients with moderate to severe ischemic stroke is thrombolytic therapy with tissue plasminogen activator (tPA), which can significantly improve neurological outcome if given within 3-4.5 hours of stroke onset. However, thrombolytic therapy is associated with an increased risk of intracerebral hemorrhage (ICH), and treatment with tPA beyond 3-4.5 hours of stroke onset further increases the risk of ICH. Understanding and preventing tPA induced hemorrhage may lead to improved and extended use of thrombolytic therapy.

Stroke is the fifth leading cause of death in the U.S. and a significant cause of adult disability. The current standard of care for patients with moderate to severe ischemic stroke is thrombolytic therapy with tissue plasminogen activator (tPA). Treatment of acute ischemic stroke with tPA can significantly improve neurological outcomes; however, thrombolytic therapy is associated with an increased risk of ICH. Due in part to the risk of hemorrhagic transformation, it is estimated that only 5-7% of ischemic stroke patients receive intravenous tPA, with another 1-2% receiving intra-arterial therapy.

Cerebral microvascular endothelial cells (ECs) help create the blood-brain barrier (BBB) through a complex and precisely regulated system of tight junction (TJ) proteins. TJs are critical for maintaining brain homeostasis by controlling permeability across the vascular endothelium. It has been shown that protein kinase C-β (PKC-β) phosphorylation of the tight junction protein occludin on Ser490 induces endothelial permeability. The experiments disclosed herein demonstrate that ischemic stroke induces phosphorylation of occludin 5490 and increased BBB permeability dependent on PKC-β activity, and that this effect is significantly attenuated in tPA^{-/-} mice. Importantly, expression of occludin mutated at Ser490 to Ala (S490A) in vascular endothelial cells of transgenic mice completely prevents ICH induced by delayed tPA administration following the induction of ischemic stroke.

Acute, ischemic stroke remains an important cause of morbidity and mortality in the United States. Each year in the U.S. there are approximately 795,000 strokes. Stroke occurs in two forms, ischemic and hemorrhagic. Approximately 87% of strokes are ischemic, while hemorrhagic stroke accounts for 13%, with about 10% resulting from ICH, and another approximately 3 percent arising from subarachnoid hemorrhage. Hemorrhagic strokes generally have a worse prognosis than ischemic stroke and are associated with high mortality. Further, the hemorrhagic conversion of an ischemic stroke can significantly increase disability and mortality. Disruption of the blood-brain barrier (BBB) is a critical pathophysiological event following cerebral ischemia, and significant increases in BBB permeability may contribute a critical role in hemorrhagic transformation.

Ischemic strokes are characterized by an abrupt onset of focal neurologic symptoms, and the prompt restoration of blood flow is the most effective approach for treating ischemic stroke. Therapies for acute ischemic stroke are limited, but intravenous delivery of recombinant tissue plasminogen activator (rtPA) within 3-4.5 hours provided better reperfusion and clinical outcome and increased survival, independence, and favorable outcome with 90 out of 1000 patients having absolute benefit¹² (Lancet Neurol., 2010, 9:866-874; N. Engl. J Med., 1995, 333:1581-1587). However, both clinical evidence (Lancet, 2004, 363:768-774; CMAJ., 2005, 172:1307-1312; Cerebrovasc. Dis., 2007, 24:1-10; Circulation, 2002, 105:1679-1685; Stroke, 1997, 28:2109-2118)^{20,22,23}, and experimental models of ischemic stroke (Nat. Med., 2008, 14:731-737)⁴, demonstrate a significant increase in the incidence of hemorrhagic transformation with late thrombolytic tPA. Therefore, understanding the molecular pathways activated by thrombolytic tPA that increase the incidence of hemorrhagic conversion could profoundly improve the treatment of ischemic stroke.

Conventional therapies for stroke have focused largely on neuroprotective compounds to maintain neuronal function in the ischemic environment induced by the stroke, but so far only rtPA has been shown to provide therapeutic benefit.

### SUMMARY

The disclosure provides methods for extending the time following the development of ischemia within which a thrombolytic compound such as tissue Plasminogen Activator (tPA) may efficaciously and safely be administered. The methods overcome the time-based limitation in using tPA, the only therapeutic available to treat stroke and other ischemic diseases. Until the disclosed methods were developed, tPA could only be administered within three, or perhaps four and one-half, hours of the onset of ischemia. Beyond that time, tPA was contra-indicated because it would deleteriously induce or promote vessel hemorrhage, or leakage. In the context of an ischemic stroke, tPA was available for treatment within three or four and one-half hours of the onset of ischemia, but beyond that time, tPA was avoided because it would increase the risk of conversion to hemorrhagic stroke, which could be fatal. The disclosed methods involve the administration of an inhibitor of a conventional Protein Kinase C isoform, i.e., PKC-α, PKC-β, or PKC-γ, before, at about the same time as, or after administration of a thrombolytic compound such as tPA, rtPA, or any of the compounds disclosed herein as providing thrombolytic activity. The administration of the two therapeutics improves the safety and extends the time window within which a thrombolytic compound is safely and efficaciously administered to a subject with an ischemic disease.

In one aspect, the disclosure provides a method of treating an ischemic disorder comprising: (a) administering a therapeutically effective amount of a thrombolytic compound; and (b) administering a therapeutically effective amount of a Protein Kinase C inhibitor, thereby treating the ischemic disorder with reduced vascular hemorrhage compared to treatment without the Protein Kinase C inhibitor. In some embodiments, the Protein Kinase C inhibitor is co-administered with the thrombolytic compound; in some embodiments, the Protein Kinase C inhibitor is administered before the thrombolytic compound is administered; and in some embodiments, the Protein Kinase C inhibitor is administered after the thrombolytic compound is administered. It is contemplated that the thrombolytic compound used in the methods according to the disclosure will partially or completely disintegrate a thrombus, or blood clot. In some embodiments, the thrombolytic compound reduces a thrombus, or partially or completely eliminates a thrombus. In some embodiments, the thrombolytic compound partially or completely dissolves the thrombus.

This aspect of the disclosure contemplates embodiments in which the ischemic disorder is stroke, cerebral ischemia, intracerebral hemorrhage, cerebral infarction, acute myocardial infarction, thrombosis, embolism, acute peripheral arterial occlusion, thoracic outlet syndrome, persistent loculated pleural fluid collection, short bowel syndrome, plastic bronchitis, kidney disease, pleural effusion, empyema, an abdominal abscess, a pelvic abscess, diabetic macular edema, or occlusion of a blood vessel access device. In some embodiments, the intracerebral hemorrhage is hypertensive intracerebral hemorrhage, ischemic cerebral accident, brain ischemia, aneurysmal subarachnoid hemorrhage, or intraventricular hemorrhage. In some embodiments, the empyema is pleural empyema. In some embodiments, the kidney disease is chronic renal insufficiency. In some embodiments, the thrombosis is deep venous thrombosis of the lower extremity. In some embodiments, the blood vessel access device is a central venous access device or an indwelling catheter. In some embodiments, the stroke is acute ischemic stroke. In some embodiments, the thrombosis is thromboembolism or deep vein thrombosis. In some embodiments, the thromboembolism is pulmonary thromboembolism. In some embodiments, the embolism is a pulmonary embolism.

This aspect of the disclosure comprehends embodiments wherein the thrombolytic compound is tissue Plasminogen Activator, recombinant tissue Plasminogen Activator, Alteplase (Activase®), Reteplase, Tenecteplase, Urokinase, Prourokinase, Anisoylated purified streptokinase activator complex (APSAC; Anistreplase), Streptokinase, Desmoteplase, Staphylokinase, or Lanoteplase. In some embodiments, the thrombolytic compound is tissue Plasminogen Activator. In some embodiments, the thrombolytic compound is recombinant tissue Plasminogen Activator. In some embodiments, the Protein Kinase C inhibitor is an inhibitor of Protein Kinase C-α, Protein Kinase C-β, or Protein Kinase C-γ. In some embodiments, the Protein Kinase C inhibitor is Ruboxistaurin®, AEB071, Aprinocarsen, Aurothioglucose hydrate, Bisindolylmaleimide II, Bisindolylmaleimide IV, Bisindolylmaleimide VII, Bisindolylmaleimide X hydrochloride, Bisindolylmaleimide XI hydrochloride, Bryostatin-1, Bryostatin 2, Bryostatin 3, Cl, Calphostin c, CGP-53353 solid, Chelerythrine chloride, n-desmethyltamoxifen HCl, Dihydrosphingosine, Enzastaurin, Flosequinan, GF109203X,Go 6976, Go 6983, Hispidin solid, Ilmofosine, Ingenol-3-angelate, K-252B solution, K252C, LY 333531 hydrochloride, LY379196, Rac-2-methoxy-3-hexadecanamido-1-propylphosphocholine, [ALA107] Myelin basic protein fragment 104-118, [ALA113]-Myelin basic protein fragment 104-118, Melittin, Midostaurin, NPC-15437 dihydrochloride hydrate, (±)-Palmitoylcarnitine chloride, PKC pseudosubstrate peptide, PKC 412, PKCβii peptide inhibitor i trifluoroacetate salt, Myristoylated Protein Kinase C inhibitor, Protein Kinase C-β pseudosubstrate, Protein Kinase C-ζ pseudosubstrate, Myristoyl trifluoroacetate salt, SBI-0087702, RO 32-0432 hydrochloride, Rottlerin, Safingol (L-threo-dyhydrosphingosine), Sotrastaurin, Staurosporine, Staurosporine ready-made solution, D-erythro-Sphingosine, Tamoxifen, Tamoxifen citrate salt, TCS 21311, 7-hydroxystaurosporine (UCN-01), or ZIP. In some embodiments, the Protein Kinase C inhibitor is Ruboxistaurin®, LY 333531, or LY379196. In some embodiments, the thrombolytic compound is administered more than three hours after the onset of ischemia. In some embodiments, the thrombolytic compound is administered more than 4.5 hours after the onset of ischemia. In some embodiments, the thrombolytic compound is administered more than five, six, seven, eight, nine, ten eleven, or twelve hours after the onset of ischemia. In some embodiments, the Protein Kinase C inhibitor is administered for at least two days. In some embodiments, the Protein Kinase C inhibitor is administered for three, four, five, six, seven, eight, nine or ten days.

Other features and advantages of the disclosure will become apparent from the following detailed description. It should be understood, however, that the detailed description and the specific examples, while indicating embodiments of the disclosed subject matter, are given by way of illustration only, because various changes and modifications within the spirit and scope of the disclosure will become apparent to those skilled in the art from this detailed description.

### BRIEF DESCRIPTION OF THE DRAWING

This patent or application file contains at least one drawing executed in color. Copies of this patent or patent application publication with color drawing(s) will be provided by the United States Patent and Trademark Office upon request and payment of the necessary fee.

FIG. 1. Schematic illustration of Occludin, a Protein Kinase C phosphorylation target, including identification of the Ser490 residue discussed in the text.

FIG. 2. (A) Relative endothelial cell permeability as a measure of vessel hemorrhage or leakage, resulting from interaction of mutant PKC with wild-type or mutant Occludin and interaction of wild-type PKC with wild-type or mutant Occludin in mice; (B) Schematic illustration of Occludin in a cell membrane; (C) Schematic illustration of the interaction of PKC-β and Occludin at the cell membrane.

FIG. 3. Confocal image of cerebral cortices of 12-week-old C57BL/6 mice subjected to 3-hour middle cerebral artery occlusion (MCAO). The penumbra region ipsilateral to the MCAO and an equivalent region contralateral to the MCAO were immunostained for basal Occludin and phosphor-Occludin specific for serine 490 (pS490).

FIG. 4. Confocal image of cerebral cortices of 12-week-old C57BL/6 mice or tPA-null mice subjected to 3-hour MCAO. (A) Confocal images of the penumbra region ipsilateral to the MCAO in wt or tPA^{-/-} mice (MCAO) and an equivalent region contralateral to the MCAO in wt mice (wt) were immunostained for basal Occludin, pS490, ZO-1 and Merged. (B) Image quantitation of pS490 immunostaining using IMARIS software in the contra- and ipsi-lateral regions

FIG. 5. PKC-β inhibitor pretreatment blocked the MCAO-induced cerebrovascular permeability and infarct size. (A) Oral administration of PKC-β inhibitor into wild-type mice reduced dextran extravasation at 24 hours post-MCAO. (B) Image quantitation of dextran leaks. (C) Total hemispheric extraction of extravasated dextran. (D) Volumetric analyses of infarct size (72 hours after MCAO). Statistics: One-Way ANOVA, *p<0.05, **p<0.01 and ***P<0.001. Data expressed as mean±SEM.

FIG. 6. MCAO-induced Occludin phosphorylation (pS490)-mediated cerebrovascular permeability in a PKC-β-dependent manner. Confocal image of cerebral cortices of 12-week-old C57BL/6 mice receiving oral administration of either vehicle (saline) or PKC-β inhibitor (LY379196) 72 hours prior to MCAO and continued daily until the completion of the experiment. Animals received an IV injection of 70 kDa dextran one hour before euthanasia. (A) Confocal images of the penumbra region ipsilateral to the MCAO (ipsi) and an equivalent region contralateral to the MCAO (Ct) of mice immunostained for basal Occludin and pS490. (B) Quantitation of pS490, basal Occludin, ZO-1 and dextran extravasation using IMARIS software in the contra- and ipsi-lateral regions. *P<0.05; ***P<0.001 (n=5); NS, not significant. Data expressed as mean±SEM.

FIG. 7. (A) A schematic illustration of the coding region for the mutant S490A Occludin in a construct suitable for Cre-Lox directed recombination. (B) Immunofluorescence photomicrograph using a fluorescently labeled P-5490-specific antibody binding following photothrombotic MCAO formation revealing a dramatic increase in OccS490 phosphorylation in the penumbra.

FIG. 8. Endothelial cell specific mutation of Occludin (S490A) reduced blood-brain barrier (BBB) leakage after MCAO. Fluorescent images of cerebral cortices of 12-week-old PDGFiCre-490A or littermate control 24 hours after MCAO. Animals received an IV injection of 70 kDa dextran one hour before euthanasia. (A) Fluorescent images of littermate control mice and PDGFiCre-490A mice 24 hours after MCAO. (B) Quantitation of dextran extravasation using IMARIS software in the contra- and ipsi-lateral regions. ***P<0.001 (n=5); data expressed as mean±SEM.

FIG. 9. Blocking Occludin phosphorylation at S490 reduced intracerebral hemorrhage associated with late tPA thrombolysis 72 hours post-MCAO. MCAO− was induced in 12-week-old wild-type (PDGFicre BP12) and serine 490-mutant mice (iCrex 490A). (A) Representative images of cerebral cortices are shown 72 hours after MCAO. (B) Intracerebral hemorrhage volume was quantified. ****P<0.001. Data expressed as mean±SEM.

FIG. 10. Blocking PKC beta reduced intracerebral hemorrhage associated with late tPA thrombolysis 72 hours post-MCAO. MCAO was induced in 12-week-old wild-type C57BL6/J mice treated with either vehicle or PKC-β inhibitor one hour post-MCAO and continued daily until the end of the experiment. Animals were also treated with either vehicle or tPA 5 hours after MCAO. Intracerebral hemorrhage volume was quantified. ***P<0.001. ****P<0.0001. Data expressed as mean±SEM.

### DETAILED DESCRIPTION

Preventing or reducing undesirable blood-brain barrier (BBB) permeability under ischemic conditions is expected to limit the damage to neural tissue in stroke and, in combination with treatment provided by a thrombolytic compound, is expected to extend clearance of thrombi while preventing loss of the BBB and hemorrhagic transformation. More generally, the methods are designed to provide an efficacious and safe therapy for ischemic diseases that expands the time window for beneficial administration of a thrombolytic compound such as tissue Plasminogen Activator or any other thrombolytic compound as disclosed herein.

“Thrombolytic compound” means any compound providing a thrombolytic activity and includes, but is not limited to, tissue Plasminogen Activator, a functional fragment of tissue Plasminogen Activator, recombinant tissue Plasminogen Activator, Alteplase (Activase®), Reteplase, Tenecteplase, Urokinase, Prourokinase, Anisoylated purified streptokinase activator complex, Streptokinase, Desmoteplase, Staphylokinase, and Lanoteplase.

“Protein Kinase C inhibitor” means a compound or molecule, including a protein, peptide or small-molecule chemical, that is capable of inhibiting an activity of Protein Kinase C such as phosphorylation catalyzed by Protein Kinase C. In particular, a “Protein Kinase C inhibitor” includes an inhibitor of a conventional Protein Kinase C such as an inhibitor of Protein Kinase C-α, Protein Kinase C-β, and/or Protein Kinase C-γ. Exemplary Protein Kinase C inhibitors include Ruboxistaurin®, AEB071, Aprinocarsen, Aurothioglucose hydrate, Bisindolylmaleimide II, Bisindolylmaleimide IV, Bisindolylmaleimide VII, Bisindolylmaleimide X hydrochloride, Bisindolylmaleimide XI hydrochloride, Bryostatin-1, Bryostatin 2, Bryostatin 3, Cl, Calphostin c, CGP-53353 solid, Chelerythrine chloride, n-desmethyltamoxifen HCl, Dihydrosphingosine, Enzastaurin, Flosequinan, GF109203X,Go 6976, Go 6983, Hispidin solid, Ilmofosine, Ingenol-3-angelate, K-252B solution, K252C, LY 333531 hydrochloride, LY379196, Rac-2-methoxy-3-hexadecanamido-1-propylphosphocholine, [ALA107] Myelin basic protein fragment 104-118, [ALA113]-Myelin basic protein fragment 104-118, Melittin, Midostaurin, NPC-15437 dihydrochloride hydrate, (±)-Palmitoylcarnitine chloride, PKC pseudosubstrate peptide, PKC 412, PKCβii peptide inhibitor i trifluoroacetate salt, Myristoylated Protein Kinase C inhibitor, Protein Kinase C-β pseudosubstrate, Protein Kinase C-ζ pseudosubstrate, Myristoyl trifluoroacetate salt, SBI-0087702, RO 32-0432 hydrochloride, Rottlerin, Safingol (L-threo-dyhydrosphingosine), Sotrastaurin, Staurosporine, Staurosporine ready-made solution, D-erythro-Sphingosine, Tamoxifen, Tamoxifen citrate salt, TCS 21311, 7-hydroxystaurosporine (UCN-01), and ZIP.

An “ischemic disease” means a condition characterized by a deleterious transient, permanent, or recurrent incidence of ischemia. Exemplary ischemic diseases include stroke (e.g., acute ischemic stroke), cerebral ischemia, intracerebral hemorrhage (e.g., hypertensive intracerebral hemorrhage, ischemic cerebral accident, brain ischemia, aneurysmal subarachnoid hemorrhage, or intraventricular hemorrhage), cerebral infarction, acute myocardial infarction, thrombosis (e.g., thromboembolism such as pulmonary thromboembolism, deep vein thrombosis, or deep venous or vein thrombosis of the lower extremity), embolism (e.g., pulmonary embolism), acute peripheral arterial occlusion, thoracic outlet syndrome, persistent loculated pleural fluid collection, short bowel syndrome, plastic bronchitis, kidney disease (e.g., chronic renal insufficiency), pleural effusion, empyema (e.g., pleural empyema), an abdominal abscess, a pelvic abscess, diabetic macular edema, or occlusion of a blood vessel access device (e.g., a central venous access device or an indwelling catheter).

Disclosed herein are data establishing that PKC inhibitors structurally similar to Ruboxistaurin® that target PKC-β reduce permeability in stroke and, importantly, prevent intracerebral hemorrhage (ICH) in mouse models of thrombolytic stroke. Further, by expressing a mutant occludin protein, the tight junction protein controlling permeability that is a target of PKC, ICH induced by administering tPA more than three hours after onset of ischemia (e.g., detection of a symptom of an ischemic disease) is prevented.

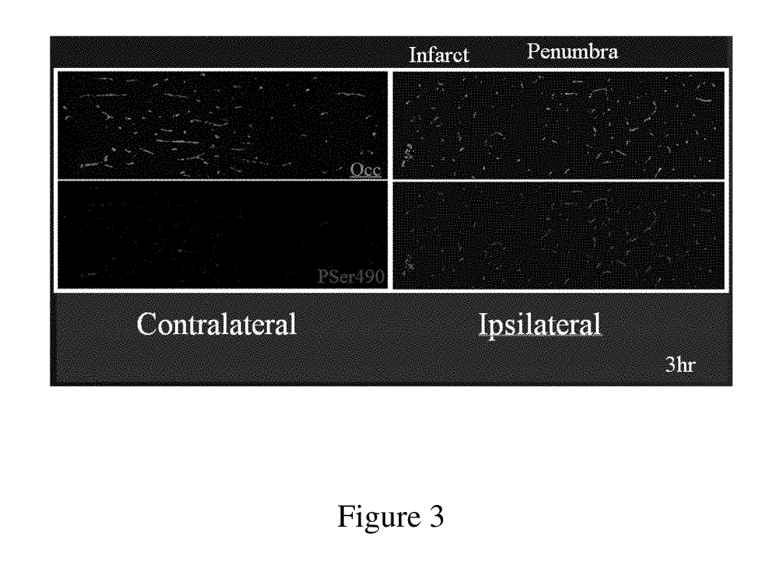

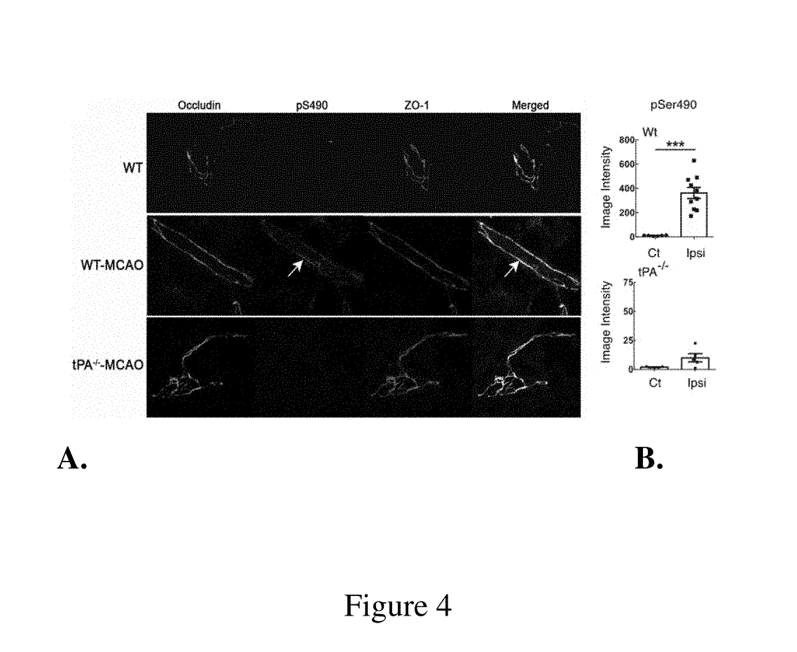

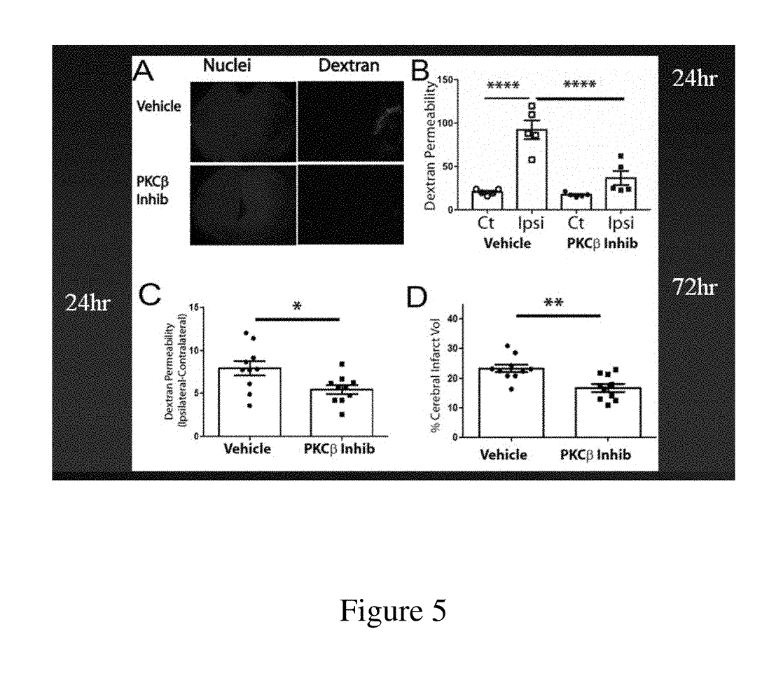

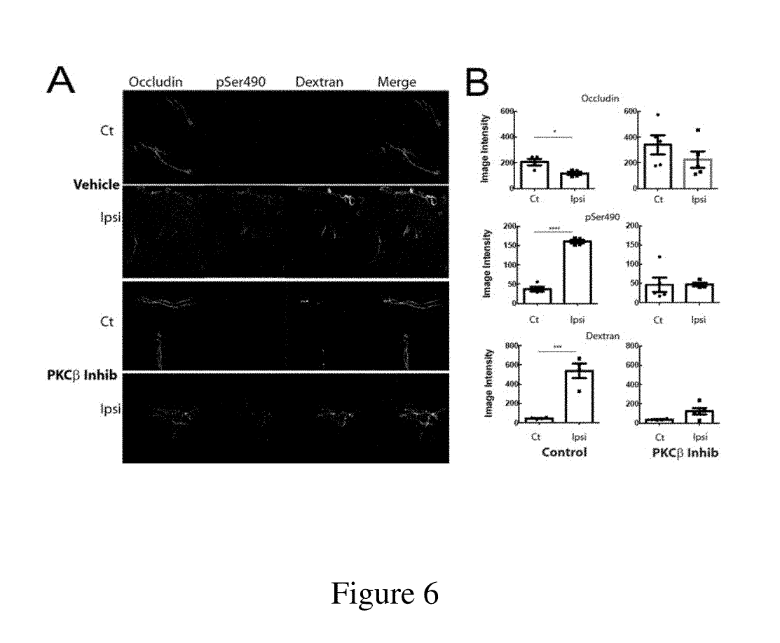

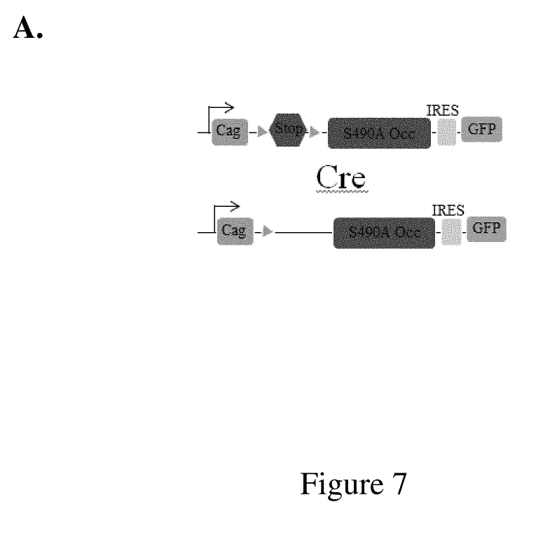

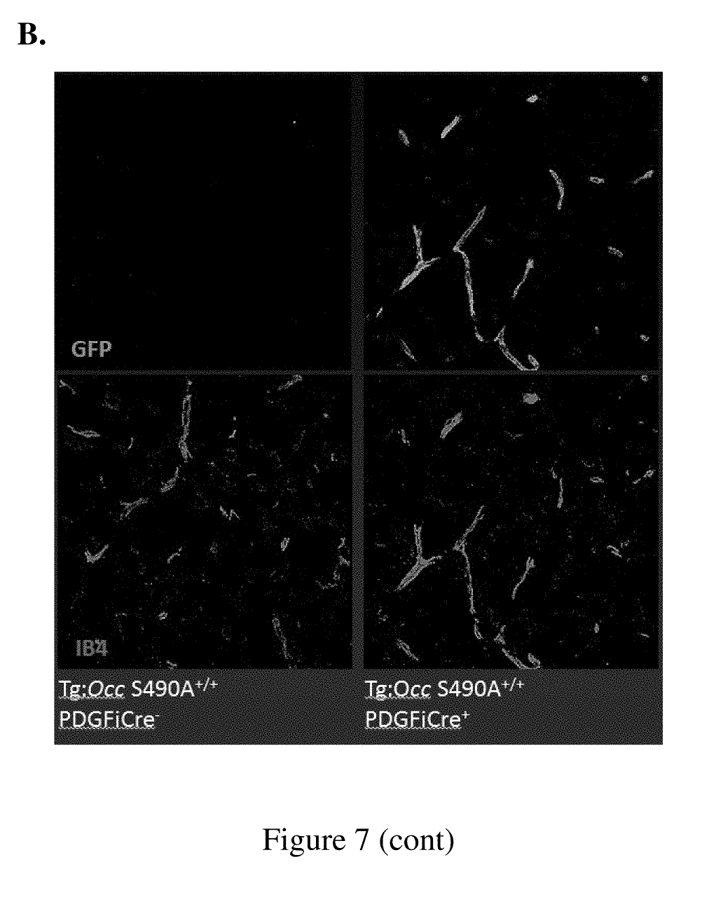

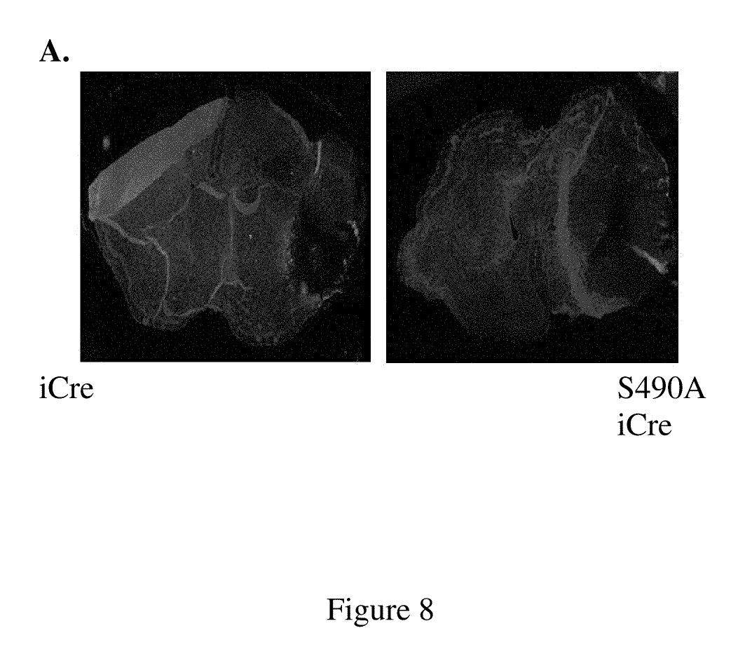

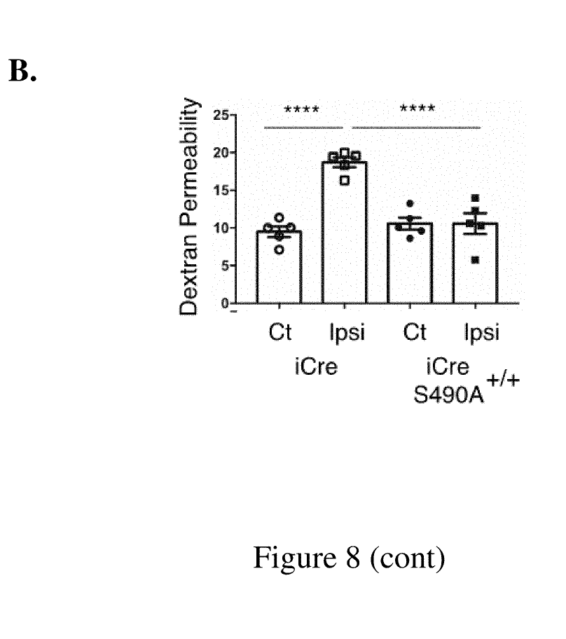

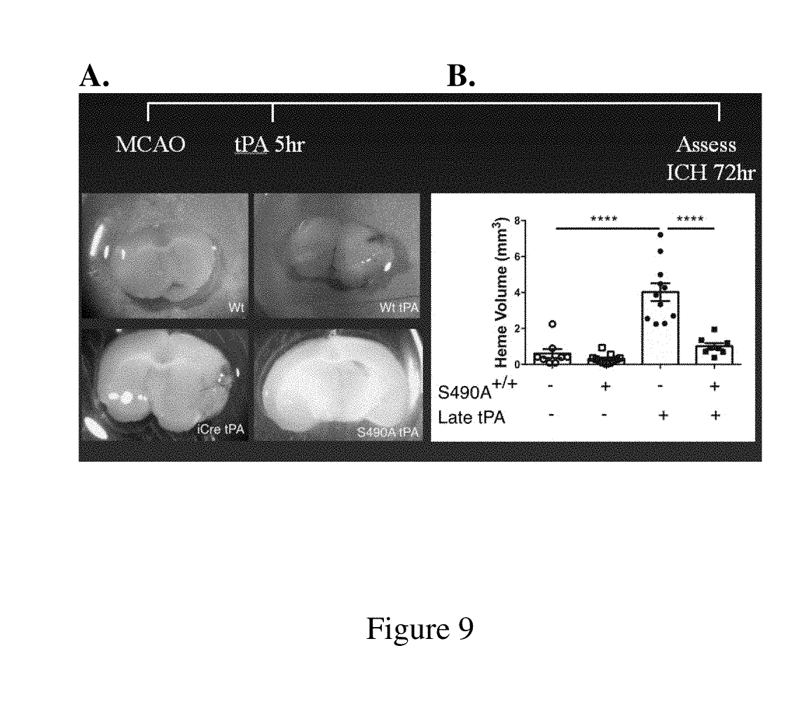

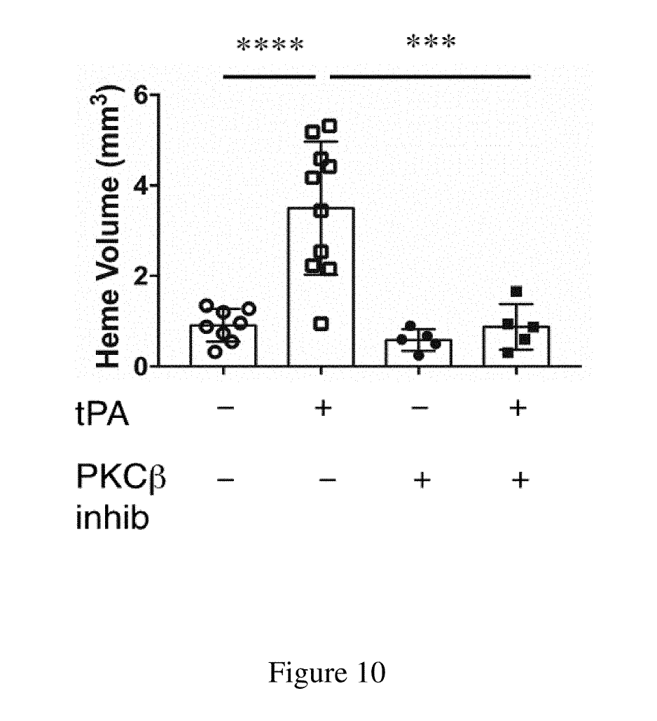

For the experiments disclosed herein, the expectation was that Ser490 phosphorylation of occludin, the target of PKC relevant to vascular permeability, would be required for tPA-induced hemorrhagic transformation in the middle cerebral artery occlusion (MCAO) model of stroke. Photothrombotic MCAO was induced in C57BL/6J mice, tPA gene deletion mice or mice carrying the occludin S490A (OccS490A) mutant at the Rosa26 locus under the CAG promoter followed by a floxed stop sequence and crossed with PDGFiCre for tamoxifen induced vascular specific expression. FIG. 7. The photothrombotic MCAO model involves exposure of a photosensitive dye, such as Rose Bengal, to light applied to the middle cerebral artery, with the illuminated dye producing singlet oxygen that damages vessel endothelia and induces occlusion. MCAO induced a dramatic increase in OccS490 phosphorylation in the penumbra, as determined by immunofluorescence microscopy using a P-S490 specific antibody. FIG. 3. MCAO in tPA-deleted animals, known to prevent an increase in vascular permeability, also blocked occludin phosphorylation. FIG. 4. Pretreatment of animals with a PKC-β inhibitor prevented the MCAO induced permeability to dextran and prevented occludin phosphorylation. FIGS. 5 and 6. Further, expression of S490A occludin also prevented MCAO-induced vascular permeability. FIG. 8. Importantly S490A mice also blocked hemorrhagic transformation in MCAO induced by late tPA thrombolysis (5 hours after MCAO). FIG. 9. PKC-β inhibitors also blocked hemorrhagic transformation induced by late tPA thrombolysis after MCAO when the kinase inhibitor was given one hour after MCAO. FIG. 10. These results provide compelling evidence that PKC-β phosphorylation of occludin Ser490 contributes to vascular permeability after MCAO and hemorrhagic transformation with late tPA treatment. The studies indicate that inhibition of PKC-β provides an opportunity to extend tPA treatment to a broader group of stroke patients.

The studies disclosed herein will provide a detailed understanding of the mechanism of ischemic stroke-induced BBB permeability changes and the contribution of tPA signaling in altering the BBB. The studies identify points of intervention to modulate permeability and have identified specific small molecule inhibitors of PKC isoforms useful in therapies to reduce ischemic stroke-induced permeability that would allow extended use of rtPA, improving outcomes after acute ischemic stroke.

All experiments described herein use randomization to treatment groups and blinded assessments. Power analysis was based on retinal ischemia reperfusion studies for changes in permeability and tight junction proteins and studies of MCAO in mouse. An n of 10 is estimated for all studies with power of 0.9 and confidence level of 95%. Data were analyzed by Student's T-test for two samples or for 3 or more groups by ANOVA followed by Turkey post-test using Graphpad (Instat) analysis software.

Transgenic mouse lines and commercially available laboratory mouse strains (Jackson Labs) were bred using standard procedures. These strains include wild-type C57BL/6J mice, and C57BL/6J mice with diet-induced obesity. Transgenic mice include mice deficient in tissue type plasminogen activator (tPA^{-/-}), mice expressing the S490A mutant of occludin, and mice expressing Cre under the vascular endothelium. The numbers of mice used are necessary to maintain the breeding colonies and to obtain statistical significance in the experiments. Mouse pups are weaned at 3 weeks of age. Mice are marked with standard mouse ear tags. Screening for transgenes is performed on DNA prepared from tail biopsies obtained using a razor blade to cut off 1 cm of the tip of the tail when the mice are 2-3 weeks old. Mice are sacrificed for collection of tissues for histological and biochemical analysis. Blood samples are obtained by cardiac puncture from anesthetized animals prior to sacrifice. Anesthesia is achieved with 450 mg/kg intraperitoneal chloral hydrate or isoflurane inhalation. For the photothromboic stroke studies, mice are placed securely under a dissecting microscope. After exposing the left middle cerebral artery (MCA), a laser Doppler flow probe (Type N (18 gauge), Transonic Systems) is attached to the surface of the cerebral cortex located 1.5 mm dorsal median from the bifurcation of MCA. Rose Bengal diluted to 2.5 mg/mL or 10 mg/mL in phosphate-buffered saline (PBS) is then injected into the tail vein with the final dose of 12.5 mg/kg or 50 mg/kg. A 1.5-mW cold green light laser (540 nm, Melles Griot) is directed at the MCA from a distance of 6 cm for 45 minutes at the onset of the injection. After occlusion, mice are allowed to recover. Both male and female mice are used, but are initially analyzed separately. If there are no gender differences, data is combined. Significant differences between genders are noted.

### EXAMPLES

### Example 1

Role of Occludin Phosphorylation in Vascular Permeability and ICH in Ischemic Stroke

It was expected that occludin phosphorylation at Ser490 would be required for occludin ubiquitination and BBB breakdown in response to late rtPA (recombinant tissue plasminogen activator administration at 5 hours after middle cerebral artery occlusion) treatment in ischemic stroke. For these studies, a mouse expressing floxed stop-S490AOcc in vascular endothelium is used. The S490A mutation completely inhibited late tPA induced ICH. Occludin S490A mice are subjected to photothrombotic middle cerebral artery occlusion (MCAO), with and without late thrombolysis (i.e., administration of rtPA) and analyzed for BBB permeability, ICH, and tight junction protein (TJ) alterations. Brain functional analysis outcomes after MCAO in the transgenic mice are also performed.

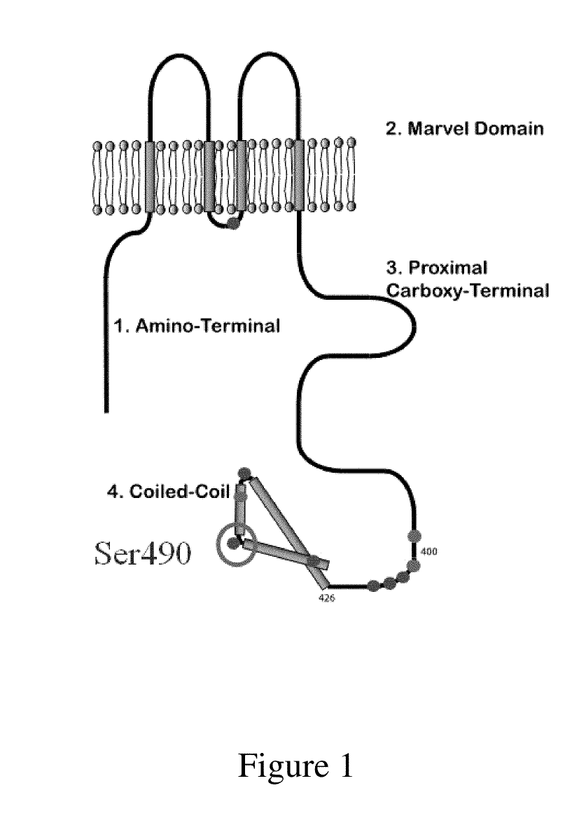

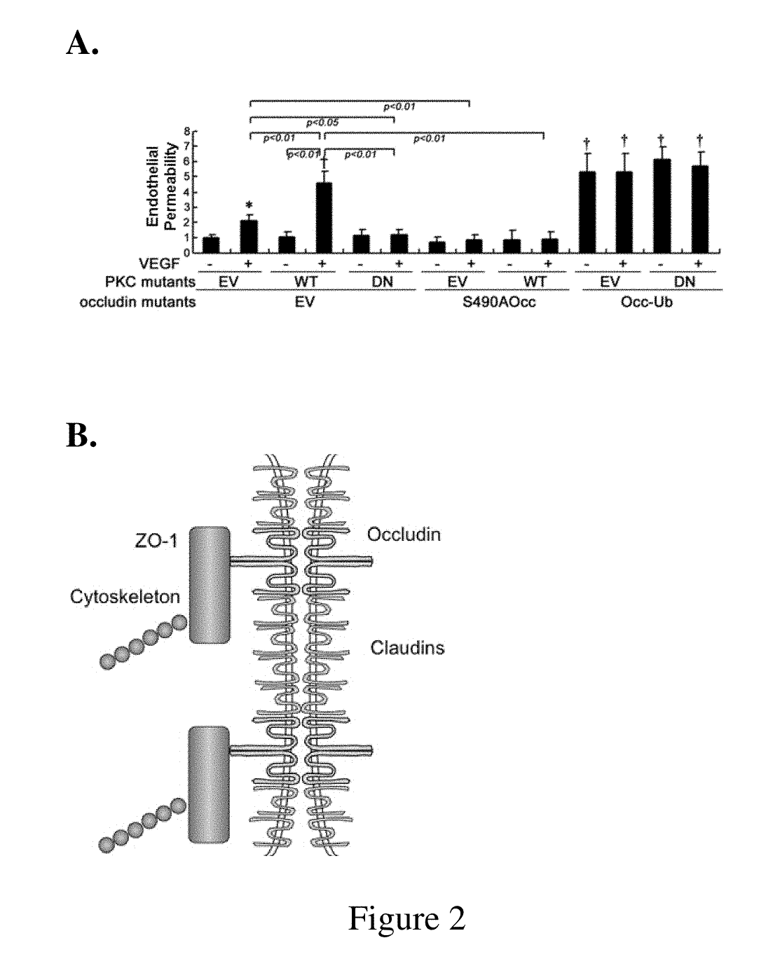

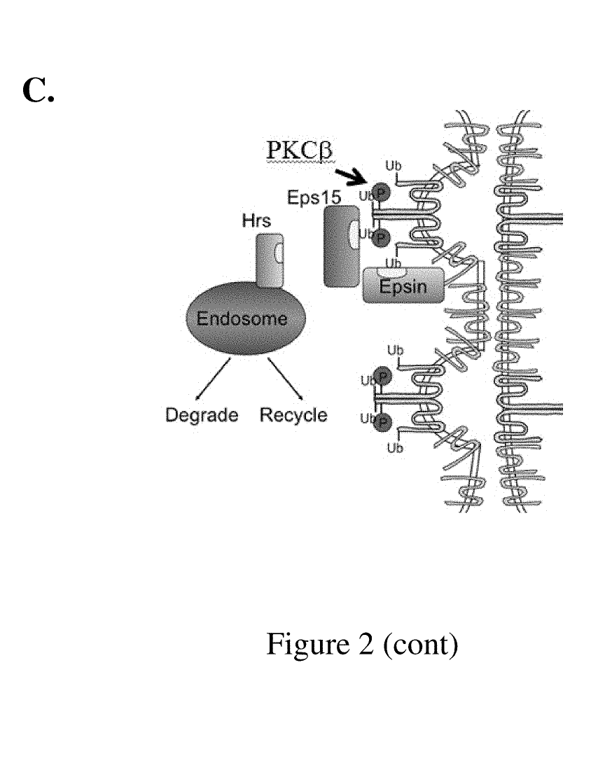

Occludin is a tetraspan tight junction protein with a coiled-coil domain in the carboxy terminus. Ser490 of occludin lies within the second turn of this coiled coil domain and adjacent to the acidic head that directly binds to the tight junction organizing protein ZO-1. FIGS. 1 and 2. The S490A mutation in occludin has been shown to inhibit phosphorylation of amino acid residue 490. PKC-β directly phosphorylates S490 on occludin, leading to subsequent ubiquitination by the ligase Itch. The ubiquitinated occludin is endocytosed through interaction with multiple chaperones and leads to tight junction protein (TJ) disassembly, with internalization of additional TJ proteins such as claudin 5 and ZO-1. Similar endocytosis of TJ leading to vascular permeability was observed in response to CCL2 (Stamatovic, et al., J. Biol. Chem. 284(28):19053-66 (2009)).

Clinical use of rtPA treatment can lessen stroke severity, but only if provided within a 3-4.5-hour window. Later treatment can lead to more severe stroke outcome with intracerrebral hemorrhage (ICH) and poor stroke outcome. Occludin phosphorylation at Ser490 is required for occludin ubiquitination and BBB breakdown in response to late rtPA treatment in ischemic stroke. Data revealed that the tight junction protein occludin is phosphorylated on Ser490 in the penumbra of mice after thrombosis. FIG. 3. By expressing the phospho-inhibitory S490A, the ICH response to late rtPA treatment (three hours post-ischemia) was prevented and MCAO-induced permeability was reduced, with the result that neurological function was improved.

To determine whether occludin phosphorylation on Ser490 increased after MCAO, mice were subjected to photothrombotic stroke using rose bengal dye, as described in (Nat. Med., 2008, 14:731-737) Brain sections were made and stained using a monoclonal occludin antibody and the phospho-Ser490 antibody previously characterized as specifically binding phospho-Ser490 occludin (J Proteome Res. 2009, 8(2):808-17). Occludin staining was apparent in the cortical vasculature in the contralateral lobe but there was very little PSer490 staining. FIG. 3. After MCAO, however, the PSer490 staining dramatically increased throughout the penumbra. FIG. 3. This analysis revealed a stark increase in Ser490 phosphorylation in the penumbra after middle cerebral artery occlusion.

To investigate the direct contribution of occludin Ser490 phosphorylation to vascular permeability, conditional expressing transgenic mice capable of expressing wild-type or the S490A phospho-inhibitory mutant of occludin were created. The occludin cDNA under a floxed stop site was integrated into the Rosa26 site of the genome using zinc-finger nuclease (ZFN) technology. A ZFN pair targeting the Rosa 26 site (Sigma) were microinjected into C57B16 mouse oocytes with plasmid, CTV vector (Addgene), containing either wild-type occludin (WtOcc) or mutant occludin (S490AOcc). The human occludin cDNA is under control of the CMV enhancer/chicken beta-actin promoter with rabbit beta-globin intron (CAG) followed by a Stop cassette that is flanked by LoxP sites, upstream of the occludin insert, allowing conditional expression by crossing with the appropriate Cre expressing mouse. The CTV vector also possessed approximately 1 kb homology to Rosa26 located 5′ to the insert and 4 kb of homology to the Rosa26 site 3′ to the insert, allowing recombination into Rosa26 after ZFN cleavage. An enhanced green fluorescent protein (eGFP) coding region was also incorporated under an internal ribosome entry site to allow easy confirmation of gene expression. GFP expression was seen in mice crossed with Tek-Cre in the brain cortex. Positive clones were selected based on construct-specific PCR and confirmed with PCR primers confirming integration into the Rosa26 site. From these results two mouse lines from both WtOcc and S490AOcc were selected for sequencing to obtain final confirmation of integrations of the occludin or mutant occludin cDNA into the Rosa26 site.

In addition, mice for conditional deletion of endogenous occludin have been obtained. Crossing Occfl/fl tgS490AOcc+/+ mice with the appropriate vascular endothelial Cre will remove endogenous occludin and allow expression of the S490A point mutant occludin. Occfl/fl tgWtOcc^{+/+} mice are used as controls to restore occludin.

Two promoters driving vascular endothelial cell-restricted Cre are used in the studies. Offspring from tgWtOcc and tgS490AOcc mice crossed with mice containing Cre under the Tek promoter (B6.Cg-Tg(Tek-Cre)1Ywaa, Jackson Labs) driving Cre expression in vascular endothelial cells are viable with normal Mendelian inheritance and appear normal. A tamoxifen-inducible, vascular specific Cre-expressing mouse is also used, which avoids Tek-driven expression in bone marrow-derived hematopoietic cells. Such a mouse has been developed that allows vascular specific expression of tamoxifen-inducible Cre (iCreERT2) under control of the platelet derived growth factor B (Pdgfb) promoter. These mice (Pdgfb-iCreER) induce lacZ expression in the vasculature of the central nervous system from the same Rosa26 locus used for the occludin constructs¹. It should be noted that both the occludin constructs and the Pdgfb-iCreERT2 mice carry eGFP under an IRES. FIG. 7. However, the conditional expression of occludin constructs can be confirmed by rtPCR. eGFP was expressed from the Tg:WtOcc and TekCre mice and Tg:S490AOcc^{+/+} from Pdgfb-iCreERT2 mice treated with tamoxifen at weaning. FIG. 7.

Studies by Shi et al. demonstrating improved vascular permeability and stroke outcome by ADFm or HSP27 expressed under the Tek promoter represent important new insights in stroke pathology (Proc Natl Acad Sci USA. 2017 Feb. 14; 114(7):E1243-E1252; Nat Commun. 2016 Jan. 27; 7:10523)). Using the Tek promoter for control of stop flox expression, however, leads to 85% of bone marrow-derived cells expressing the gene as well as endothelial cells (Genesis. 2010 September; 48(9):563-7)). Further, changing actin polymerization may have broader effects than just an effect on vascular tight junctions alone. Pdgfb-iCreERT2 mice that yield a highly restrictive vascular endothelial expression³¹ (Genesis. 2008 February; 46(2):74-80) were used for conditional expression of the tight junction protein occludin mutant Tg:S490AOcc^{+/+}. Expression of Tg:S490AOcc^{+/+} reduced permeability induced by MCAO, as measured using 70 kDa dextran labeled with Texas Red (dextran). MCAO was induced in control Cre⁺ mice or Tg:S490AOcc^{+/+} Cre⁺ and 24 hours later dye was perfused to measure permeability. Brains were removed, fixed, and imaged for dextran red. Five images surrounding the infarct were taken and mean pixel intensity was determined using Imaris software, averaged for each mouse with n of at least 8 mice. Results showed a clear increase in permeability from MCAO compared to the control, contralateral side. However, expression of Tg:S490AOcc^{+/+} prevented the increase in permeability. In separate experiments, mice were subjected to stroke induction and then given rtPA 5 hours after stroke. A clear increase in hemorrhagic transformation from this late rtPA treatment was observed, as measured by quantifying the area of hemoglobin in serial brain sections. FIG. 9. However, expression of Tg:S490AOcc^{+/+} completely prevented this ICH, compared to control Cre⁺ only mice. FIG. 9.

### Example 2

Protein Kinase C Reduced Vascular Permeability and ICH in Ischemic Stroke

PKC isoforms are divided into three basic groups. The conventional PKCs (cPKC) require elevated calcium and diacylglycerol as cofactors while the novel PKC isotypes require diacylglycerol alone and atypical PKC is activated downstream of phosphatidyl inositol 3-kinase. The conventional PKCs, i.e., PKC-α, PKC-β and PKC-γ, are contemplated as targets for inhibition in extending the window for treatment of an ischemic disease with a thrombolytic compound.

PKC signaling contributes to BBB permeability. Therefore, experiments were designed to determine if inhibition of PKC-β is effective at reducing MCAO-induced permeability and tPA-associated hemorrhagic transformation. For these studies, wild-type (WT) mice were subjected to MCAO, with or without late thrombolysis (i.e., administration of a tPA compound at least 5 hours after MCAO), treated with a specific inhibitor of PKC-β, and analyzed for cerebrovascular permeability, ICH, and TJ alterations as well as measures of functional outcomes.

Experimental results reveal that blocking phosphorylation of the occludin Ser490 target of PKC prevented MCAO-induced permeability and MCAO-induced, and late rtPA-induced, ICH. Therefore, it is expected that any PKC-β inhibition will reduce permeability in MCAO and prevent ICH after late rtPA. Ruboxistaurin and other compounds inhibiting at least one conventional PKC are expected to be effective therapies for ischemic stroke, which may only require acute dosing, particularly to prevent tPA-induced permeability and ICH. In addition, it is expected that such cPKC inhibitors will allow a longer treatment window for tPA administration to treat ischemic diseases such as stroke.

Inhibition of PKC-β reduces MCAO induced cerebral vascular permeability. The PKC-β specific inhibitor LY333531 (Tocris) was delivered by gavage at 10 mg/kg for three doses over 3 days. Photothrombotic MCAO was induced and permeability to fluorescent-labeled 70 kDa dextran was measured 24 hours later in brain sections, as described herein, along with PS490 phosphorylation. FIG. 5 reveals BBB permeability to Texas Red dextran after MCAO with PKC-β inhibitor pretreatment providing a near complete block of dye released into the neural parenchyma of the penumbra in the cross section examination. In addition, the PKC-β inhibitor also blocked the pS490 occludin phosphorylation observed after MCAO in the penumbra. In separate experiments, providing the PKC-β inhibitor and performing surgical removal of the ischemic region and penumbra followed by dye extraction revealed an approximate 30% reduction in cerebral permeability to dextran, strongly indicating an important role for PKC-β in MCAO-induced permeability. Finally, the PKC-β inhibitor led to a significant reduction in cerebral infarct volume as measured by triphenyltetrazolium chloride (TTC) dye staining.

In addition to these studies, the effect of the PKC-β inhibition on hemorrhagic transformation was measured after tPA induction in MCAO mice. MCAO was induced and PKC-β inhibitor was given one hour later and rtPA was given 5 hours after MCAO. As observed with S490A mice, the PKC-β inhibitor led to a near complete prevention of hemorrhage in the brain after MCAO. FIG. 10. The results indicate that PKC phosphorylation of occludin on Ser490 is a required step in tPA-induced hemorrhagic transformation. The availability of PKC-β inhibitors provides a treatment paradigm to allow extended use of tPA in stroke patients without hemorrhagic transformation.

The contribution of PKC-β signaling to MCAO permeability is assessed in C57B16 mice. First, activity of conventional PKCs (cPKC), i.e., PKC-α, PKC-β, and PKC-γ, are measured in samples of cerebral tissue after MCAO. PKC activity is measured using a targeted ELISA (Abcam PKC Kinase Activity Assay) that makes use of a PKC-specific peptide substrate pre-coated on the well of a microplate and phospho-peptide-specific antibody for detection and quantification. The same time course after MCAO is measured as was used for occludin phosphorylation and stroke regions are compared to the contralateral side. In addition, immunoprecipitation of specific PKC isoforms is carried out and blotted with phospho-specific antibodies to detect activation through autophosphorylation sites such as phospho-Thr 641 for PKC-β II. Next, the effect of inhibition of PKC-β with LY333531 (Tocris) pretreatment is tested. Drug is delivered by gavage at 10 mg/kg for three or six doses over 3 days or 6 days. Vascular permeability to Evan's blue dye or Texas Red 70 kDa dextran is determined 3 hours or 24 hours after MCAO induction and infarct size is determined 3 days after MCAO. FIG. 5. In separate studies, the PKC-β inhibitor treatment is initiated one hour after MCAO and continued daily for 3 days. rtPA was then delivered at 5 hours after MCAO, and hemorrhagic transformation was determined and compared to control animals without PKC-β inhibitor 72 hours after MCAO. FIG. 10.

For all study paradigms, immunocytochemistry and Western blotting measured changes to the TJ complex in the cerebral vasculature and Ser490 phosphorylation. The effectiveness of PKC-β inhibition in preventing neurologic deficit is carried out both after MCAO and after MCAO with late (at least three hours post-ischemia) rtPA treatment.

The response to intra-cerebroventricular injection of rtPA is determined on vascular permeability and PKC-β signaling. PKC activity is measured as above after rtPA intra-ventricular injection. The PKC-β inhibitor is delivered simultaneously with rtPA by intra-ventricular injection and permeability 24 hours later is determined and compared to mice with only rtPA administration. The response to permeability, and occludin phosphorylation, are determined as described herein. These studies establish the contribution of cPKC activation to occludin phosphorylation and BBB permeability through local inhibitor delivery.

Collectively, these studies establish the contribution of cPKC to permeability after MCAO and after direct rtPA addition. It is expected that PKC-β inhibition prevents tPA-induced permeability, allowing longer tPA treatment windows.

A conservative approach has been taken in describing the above experiments in the present tense as prophetic examples, although some of the described experiments have been performed. Results reveal that PKC-β inhibitor reduces permeability after MCAO and blocks hemorrhagic transformation after MCAO with rtPA. This treatment at least partially normalizes the neurologic dysfunction associated with MCAO. The PKC-β inhibitor has also been shown to block tPA-induced permeability. The cPKC inhibitor blocked tPA-induced permeability, establishing these compounds as therapeutic agents that extend the time window of treatment of ischemic disease with rtPA.

### REFERENCES

(1) Kochanek K D, Murphy S L, Xu J, Tejada-Vera B. Deaths: Final Data for 2014. Natl Vital Stat Rep 2016; 65:1-122.
(2) Prabhakaran S, Ruff I, Bernstein R A. Acute stroke intervention: a systematic review. JAMA 2015; 313:1451-62.
(3) Kleindorfer D, de los Rios La Rosa, Khatri P, Kissela B, Mackey J, Adeoye O. Temporal trends in acute stroke management. Stroke 2013; 44:S129-S131.
(4) Su E J, Fredriksson L, Geyer M et al. Activation of PDGF-CC by tissue plasminogen activator impairs blood-brain barrier integrity during ischemic stroke. Nat Med 2008; 14:731-7.
(5) Lewandowski S A, Nilsson I, Fredriksson L et al. Presymptomatic activation of the PDGF-CC pathway accelerates onset of ALS neurodegeneration. Acta Neuropathol 2016; 131:453-64.
(6) Fredriksson L, Lawrence D A, Medcalf R L. tPA Modulation of the Blood-Brain Barrier: A Unifying Explanation for the Pleiotropic Effects of tPA in the CNS. Semin Thromb Hemost 2017; 43:154-68.
(7) Wahlgren N, Thoren M, Hojeberg B et al. Randomized assessment of imatinib in patients with acute ischaemic stroke treated with intravenous thrombolysis. J Intern Med 2016.
(8) Fredriksson L, Stevenson T, Su E et al. Identification of a neurovascular signaling pathway regulating seizures in mice. Annals of Clinical and Translational Neurology 2015; 2:722-38.
(9) Mozaffarian D, Benjamin E J, Go A S et al. Heart Disease and Stroke Statistics-2016 Update: A Report From the American Heart Association. Circulation 2016; 133:e38-360.
(10) Keep R F, Hua Y, Xi G. Intracerebral hemorrhage: mechanisms of injury and therapeutic targets. Lancet Neurol 2012; 11:720-31.
(11) The NINDS t-PA Stroke Study Group. Intracerebral hemorrhage after intravenous t-PA therapy for ischemic stroke. Stroke 1997; 28:2109-18.
(12) Tanne D, Kasner S E, Demchuk A M et al. Markers of increased risk of intracerebral hemorrhage after intravenous recombinant tissue plasminogen activator therapy for acute ischemic stroke in clinical practice: the Multicenter rt-PA Stroke Survey. Circulation 2002; 105:1679-85.
(13) Hacke W, Donnan G, Fieschi C et al. Association of outcome with early stroke treatment: pooled analysis of ATLANTIS, ECASS, and NINDS rt-PA stroke trials. Lancet 2004; 363:768-74.
(14) Hill M D, Buchan A M. Thrombolysis for acute ischemic stroke: results of the Canadian Alteplase for Stroke Effectiveness Study. CMAJ 2005; 172:1307-12.
(15) Lansberg M G, Albers G W, Wijman C A. Symptomatic intracerebral hemorrhage following thrombolytic therapy for acute ischemic stroke: a review of the risk factors. Cerebrovasc Dis 2007; 24:1-10.
(16) Latour L L, Kang D W, Ezzeddine M A, Chalela J A, Warach S. Early blood-brain barrier disruption in human focal brain ischemia. Ann Neurol 2004; 56:468-77.
(17) Sandoval K E, Witt K A. Blood-brain barrier tight junction permeability and ischemic stroke. Neurobiol Dis 2008; 32:200-19.
(18) Kassner A, Merali Z. Assessment of Blood-Brain Barrier Disruption in Stroke. Stroke 2015; 46:3310-5.
(19) Merali Z, Huang K, Mikulis D, Silver F, Kassner A. Evolution of blood-brain-barrier permeability after acute ischemic stroke. PLoS One 2017; 12:e0171558.
(20) Sandercock P, Wardlaw J M, Lindley R I et al. The benefits and harms of intravenous thrombolysis with recombinant tissue plasminogen activator within 6 h of acute ischaemic stroke (the third international stroke trial [IST-3]): a randomised controlled trial. Lancet 2012; 379:2352-63.
(21) Wahlgren N, Moreira T, Michel P et al. Mechanical thrombectomy in acute ischemic stroke: Consensus statement by ESO-Karolinska Stroke Update 2014/2015, supported by ESO, ESMINT, ESNR and EAN. Int J Stroke 2016; 11:134-47.
(22) The National Institute of Neurological Disorders and Stroke rt-PA Stroke Study Group. Tissue plasminogen activator for acute ischemic stroke. N Engl J Med 1995; 333:1581-7.
(23) Ahmed N, Wahlgren N, Grond M et al. Implementation and outcome of thrombolysis with alteplase 3-4.5 h after an acute stroke: an updated analysis from SITS-ISTR. Lancet Neurol 2010; 9:866-74.
(24) Lapchak P A, Chapman D F, Zivin J A. Metalloproteinase inhibition reduces thrombolytic (tissue plasminogen activator)-induced hemorrhage after thromboembolic stroke. Stroke 2000; 31:3034-40.
(25) Fan X, Qiu J, Yu Z et al. A rat model of studying tissue-type plasminogen activator thrombolysis in ischemic stroke with diabetes. Stroke 2012; 43:567-70.
(26) Su E J, Fredriksson L, Schielke G P, Eriksson U, Lawrence D A. Tissue plasminogen activator-mediated PDGF signaling and neurovascular coupling in stroke. J Thromb Haemost 2009; 7 Suppl 1:155-8.
(27) Fredriksson L, Li H, Fieber C, Li X, Eriksson U. Tissue plasminogen activator is a potent activator of PDGF-CC. EMBO J 2004; 23:3793-802.
(28) Fredriksson L, Ehnman M, Fieber C, Eriksson U. Structural requirements for activation of latent platelet-derived growth factor C C by tissue plasminogen activator. J Biol Chem 2005; 280:26856-62.
(29) Rodriguez-Gonzalez R, Blanco M, Rodriguez-Yanez M, Moldes O, Castillo J, Sobrino T. Platelet derived growth factor-C C isoform is associated with hemorrhagic transformation in ischemic stroke patients treated with tissue plasminogen activator. Atherosclerosis 2013; 226:165-71.
(30) Merali Z, Leung J, Mikulis D, Silver F, Kassner A. Longitudinal assessment of imatinib's effect on the blood-brain barrier after ischemia/reperfusion injury with permeability MRI. Transl Stroke Res 2015; 6:39-49.
(31) Claxton S, Kostourou V, Jadeja S, Chambon P, Hodivala-Dilke K and Fruttiger M. Efficient, inducible Cre-recombinase activation in vascular endothelium. Genesis. 2008; 46:74-80.
(32) Hua Y, Schallert T, Keep R F, Wu J, Hoff J T and Xi G. Behavioral tests after intracerebral hemorrhage in the rat. Stroke. 2002; 33:2478-84.
(33) Nakamura T, Xi G, Hua Y, Schallert T, Hoff J T and Keep R F. Intracerebral hemorrhage in mice: model characterization and application for genetically modified mice. J Cereb Blood Flow Metab. 2004; 24:487-94.
(34) Su E J, Fredriksson L, Geyer M, Folestad E, Cale J, Andrae J, Gao Y, Pietras K, Mann K, Yepes M, Strickland D K, Betsholtz C, Eriksson U and Lawrence D A. Activation of PDGF-CC by tissue plasminogen activator impairs blood-brain barrier integrity during ischemic stroke. Nature Medicine. 2008; 14:731-7.
(35) Campbell M, Hanrahan F, Gobbo O L, Kelly M E, Kiang A S, Humphries M M, Nguyen A T, Ozaki E, Keaney J, Blau C W, Kerskens C M, Cahalan S D, Callanan J J, Wallace E, Grant G A, Doherty C P and Humphries P. Targeted suppression of claudin-5 decreases cerebral oedema and improves cognitive outcome following traumatic brain injury. Nat Commun. 2012; 3:849.
(36) Harhaj N S, Felinski E A, Wolpert E B, Sundstrom J M, Gardner T W and Antonetti D A. VEGF activation of protein kinase C stimulates occludin phosphorylation and contributes to endothelial permeability. Invest Ophthalmol Vis Sci. 2006; 47:5106-15.
(37) Murakami T, Frey T, Lin C and Antonetti D A. Protein kinase cbeta phosphorylates occludin regulating tight junction trafficking in vascular endothelial growth factor-induced permeability in vivo. Diabetes. 2012; 61:1573-83.
(38) Aiello L P, Vignati L, Sheetz M J, Zhi X, Girach A, Davis M D, Wolka A M, Shahri N and Milton R C. Oral protein kinase c beta inhibition using ruboxistaurin: efficacy, safety, and causes of vision loss among 813 patients (1,392 eyes) with diabetic retinopathy in the Protein Kinase C beta Inhibitor-Diabetic Retinopathy Study and the Protein Kinase C beta Inhibitor-Diabetic Retinopathy Study 2. Retina. 2011; 31:2084-94.
(39) Strom C, Sander B, Klemp K, Aiello L P, Lund-Andersen H and Larsen M. Effect of ruboxistaurin on blood-retinal barrier permeability in relation to severity of leakage in diabetic macular edema. Invest Ophthalmol Vis Sci. 2005; 46:3855-8.
(40) Cipolla M J, Huang Q and Sweet J G. Inhibition of protein kinase Cbeta reverses increased blood-brain barrier permeability during hyperglycemic stroke and prevents edema formation in vivo. Stroke. 2011; 42:3252-7.

Each of the references cited herein is hereby incorporated by reference in its entirety or in relevant part, as would be apparent from the context of the citation.

From the disclosure herein it will be appreciated that, although specific embodiments of the disclosure have been described herein for purposes of illustration, various modifications may be made without deviating from the spirit and scope of the disclosure.

What is claimed is:

1. A method of treating an ischemic disorder comprising:
(a) administering a therapeutically effective amount of a thrombolytic compound; and
(b) administering a therapeutically effective amount of a Protein Kinase C inhibitor, thereby treating the ischemic disorder with reduced vascular hemorrhage compared to treatment without the Protein Kinase C inhibitor.

2. The method of claim 1 wherein the Protein Kinase C inhibitor is co-administered with the thrombolytic compound.

3. The method of claim 1 wherein the Protein Kinase C inhibitor is administered before the thrombolytic compound is administered.

4. The method of claim 1 wherein the Protein Kinase C inhibitor is administered after the thrombolytic compound is administered.

5. The method of claim 1 wherein the ischemic disorder is stroke, cerebral ischemia, intracerebral hemorrhage, cerebral infarction, acute myocardial infarction, thrombosis, embolism, acute peripheral arterial occlusion, thoracic outlet syndrome, persistent loculated pleural fluid collection, short bowel syndrome, plastic bronchitis, kidney disease, pleural effusion, empyema, an abdominal abscess, a pelvic abscess, diabetic macular edema, or occlusion of a blood vessel access device.

6. The method of claim 5 wherein the intracerebral hemorrhage is hypertensive intracerebral hemorrhage, ischemic cerebral accident, brain ischemia, aneurysmal subarachnoid hemorrhage, or intraventricular hemorrhage.

7. The method of claim 5 wherein the empyema is pleural empyema.

8. The method of claim 5 wherein the kidney disease is chronic renal insufficiency.

9. The method of claim 5 wherein the thrombosis is deep venous thrombosis of the lower extremity.

10. The method of claim 5 wherein the blood vessel access device is a central venous access device or an indwelling catheter.

11. The method of claim 5 wherein the stroke is acute ischemic stroke.

12. The method of claim 5 wherein the thrombosis is thromboembolism or deep vein thrombosis.

13. The method of claim 12 wherein the thromboembolism is pulmonary thromboembolism.

14. The method of claim 5 wherein the embolism is a pulmonary embolism.

15. The method of claim 1 wherein the thrombolytic compound is tissue Plasminogen Activator, recombinant tissue Plasminogen Activator, Alteplase, Reteplase, Tenecteplase, Urokinase, Prourokinase, Anisoylated purified streptokinase activator complex, Streptokinase, Desmoteplase, Staphylokinase, or Lanoteplase.

16. The method of claim 15 wherein the thrombolytic compound is tissue Plasminogen Activator.

17. The method of claim 16 wherein the tissue Plasminogen Activator is recombinant tissue Plasminogen Activator.

18. The method of claim 1 wherein the Protein Kinase C inhibitor is an inhibitor of Protein Kinase C-α, Protein Kinase C-β, or Protein Kinase C-γ.

19. The method of claim 1 wherein the Protein Kinase C inhibitor is Ruboxistaurin®, AEB071, Aprinocarsen, Aurothioglucose hydrate, Bisindolylmaleimide II, Bisindolylmaleimide IV, Bisindolylmaleimide VII, Bisindolylmaleimide X hydrochloride, Bisindolylmaleimide XI hydrochloride, Bryostatin-1, Bryostatin 2, Bryostatin 3, Cl, Calphostin c, CGP-53353 solid, Chelerythrine chloride, n-desmethyltamoxifen HCl, Dihydrosphingosine, Enzastaurin, Flosequinan, GF109203X,Go 6976, Go 6983, Hispidin solid, Ilmofosine, Ingenol-3-angelate, K-252B solution, K252C, LY 333531 hydrochloride, LY379196, Rac-2-methoxy-3-hexadecanamido-1-propylphosphocholine, [ALA107] Myelin basic protein fragment 104-118, [ALA113]-Myelin basic protein fragment 104-118, Melittin, Midostaurin, NPC-15437 dihydrochloride hydrate, (±)-Palmitoylcarnitine chloride, PKC pseudosubstrate peptide, PKC 412, PKCβii peptide inhibitor i trifluoroacetate salt, Myristoylated Protein Kinase C inhibitor, Protein Kinase C-β pseudosubstrate, Protein Kinase C-ζ pseudosubstrate, Myristoyl trifluoroacetate salt, SBI-0087702, RO 32-0432 hydrochloride, Rottlerin, Safingol (L-threo-dyhydrosphingosine), Sotrastaurin, Staurosporine, Staurosporine ready-made solution, D-erythro-Sphingosine, Tamoxifen, Tamoxifen citrate salt, TCS 21311, 7-hydroxystaurosporine (UCN-01), or ZIP.

20. The method of claim 1 wherein the Protein Kinase C inhibitor is Ruboxistaurin®, LY 333531, or LY379196.

21. The method of claim 1 wherein the thrombolytic compound is administered more than three hours after the onset of ischemia.

22. The method of claim 21 wherein the thrombolytic compound is administered more than 4.5 hours after the onset of ischemia.

23. The method of claim 21 wherein the thrombolytic compound is administered more than five, six, seven, eight, nine, ten eleven, or twelve hours after the onset of ischemia.

24. The method of claim 1 wherein the Protein Kinase C inhibitor is administered for at least two days.

25. The method of claim 24 wherein the Protein Kinase C inhibitor is administered for three, four, five, six, seven, eight, nine or ten days.
